(19) United States
(12) Patent Application Publication
(10) Pub. No.: US 2013/0253358 A1
(43) Pub. Date: Sep. 26, 2013

(54) SYSTEM AND METHOD FOR REMOTE COLLECTION AND ANALYSIS OF VOLATILE ORGANIC COMPONENTS IN BREATH

(71) Applicant: MICHAEL PHILLIPS, FORT LEE, NJ (US)

(72) Inventor: MICHAEL PHILLIPS, FORT LEE, NJ (US)

(73) Assignee: MENSSANA RESEARCH, INC., FORT LEE, NJ (US)

(21) Appl. No.: 13/786,921

(22) Filed: Mar. 6, 2013

Related U.S. Application Data
(60) Provisional application No. 61/607,775, filed on Mar. 7, 2012.

(51) Int. Cl. G01N 33/497

(52) U.S. Cl. CPC G01N 33/497
USPC 600/532

(57) ABSTRACT

A tool for telemedicine including an improved breath collection system of human breath to facilitate the analysis of volatile organic components (VOCs) contained in human breath in which breath tests can be performed at remote sites for rapid detection of different diseases. The system can include a standoff breath collection device including an arcuate structure for concentration and analysis of volatile organic components (VOCs) at the point-of-use that avoids the use of mouthpieces found in conventional breath collection apparatuses.

### CROSS REFERENCE TO RELATED APPLICATION

This application claims the benefit of U.S. Provisional Patent Application No. 61/607,775 filed Mar. 7, 2012, the entirety of which is hereby incorporated by reference into this application.

### FIELD OF THE INVENTION

The present invention relates a new tool for telemedicine including an improved breath collection system of human breath to facilitate the analysis of volatile organic components (VOCs) contained in human breath in which breath tests can be performed at remote sites for rapid detection of different diseases. The invention can employ a standoff technique for the collection of a breath sample that eliminates the need for a donor to breathe into a mouthpiece.

### BACKGROUND OF THE INVENTION

The early history of breath testing: In the 18^{th} century, Lavoisier developed a breath test for carbon dioxide in the breath. This was the first chemical probe of metabolism, and it provided the first evidence that foodstuffs are oxidized in the body. During the 19^{th} century, colorimetric breath tests detected ethanol in alcohol drinkers, and acetone in diabetics with ketoacidosis. The introduction of radio-labeled drugs in the 20^{th} century led to new breath tests for digestive disorders including Helicobacter pylori infection and pancreatic insufficiency.

The first microanalysis of breath volatile organic components (VOCs) was reported by Linus Pauling in 1971. He froze breath volatile organic components (VOCs) in a tube chilled in acetone and dry ice, and analyzed the concentrated sample using the then-new technology of GC. This resulted in the remarkable discovery that normal human breath contains a large number of volatile organic components (VOCs) in very low concentrations. Subsequent studies have shown that a sample of human breath contains several hundred different volatile organic components (VOCs), most of them in picomolar (10⁻¹² M) concentrations.

Limitations of early breath testing technology: Initially, breath microanalysis could only be performed in specialized laboratories. Pauling's pioneering research employed bulky and expensive bench-top instruments for breath volatile organic components (VOCs) microanalysis. The first clinical studies of breath biomarkers of lung cancer during the 1980s required patients to donate breath samples in a laboratory. The main technical challenge is sample collection: while it is simple to inflate a plastic bag with breath, the samples are usually too contaminated to detect breath volatile organic components (VOCs) in picomolar concentrations. The main sources of error in breath sample collection: chemical contamination, resistance to expiration, water condensation, dead-space air dilution, and container adsorption artifact 3

Advances in laboratory-based breath volatile organic components (VOCs) analysis: Progress in clinical applications of breath testing was slow until the development of the breath collection apparatus (BCA) that enabled breath volatile organic components (VOCs) sample collections in the field via a subject breathing into a mouth piece. Breath volatile organic components (VOCs) samples are captured onto sorbent traps that are sealed hermetically and sent to the laboratory for analysis by benchtop instruments e.g., automated thermal desorption with gas chromatography and mass spectroscopy (ATD/GC/MS). This technique is useful for biomarker discovery, but its value in clinical practice is limited by the high cost of the instrumentation, and the comparatively slow turnaround time of a laboratory-based procedure.

Clinical applications of breath testing: BCA technology made it possible, for the first time, to perform large multicenter studies of breath testing. These studies have demonstrated that breath volatile organic components (VOCs) contain sensitive and specific biomarkers of different diseases.

Based on the above, the breath of humans and animals contains a large number of volatile organic components (VOCs). Many of these volatile organic components (VOCs) are now recognized as biomarkers of disease, so that a breath test for volatile organic components (VOCs) offers a safe and non-invasive approach to disease detection. However, this presents several technical difficulties. The most abundant breath volatile organic components (VOCs) (e.g., acetone, isoprene, and pentane) are present in nanomolar concentrations (10⁻⁹ mol/L) but the majority of breath volatile organic components (VOCs) are present in much lower concentrations, e.g., picomolar (10⁻¹² moll), or even as low as parts per trillion. These concentrations are often below the lower limits of detection of most laboratory instruments in current use. As a result, the breath sample of interest must be collected and concentrated prior to assay. In addition, there are concomitant risks of artifactual contamination of the breath sample during the collection process. Consequently, there is a need for a breath collection system that overcomes these difficulties and delivers test results rapidly, and can be employed at the point-of-care to collect, concentrate and analyze breath volatile organic components (VOCs), transmit the data expeditiously to a central site for analytical interpretation of the data and to send the results of the breath test to the point-of-care.

### SUMMARY OF THE INVENTION

The present invention relates to a telemedicine system and method for remote collection and analysis of volatile organic components (VOCs) in breath in which breath tests can be performed at a point-of-care system and analysis of collected data can be performed at a central site for rapid detection of different diseases. The central site can include processing for interpretation of analytic data from the point-of-care system to identify if a pattern of volatile organic components (VOCs) in the sample is consistent with a particular disease. Example diseases include active pulmonary tuberculosis, lung cancer, or breast cancer. A plurality of point-of-care systems can interface with the central site. Algorithm learning can be used at the central site to refine diagnostic algorithms used in interpretation of the analytic data to improve their accuracy based on accumulated data from collected breath samples at the point-of-care system in an ongoing fashion.

Reports can be generated at the central site and can include an interpretation of high or low risk of a disease, such as active pulmonary tuberculosis. The reports can be transmitted to the point-of-care instrument.

The central site can provide remote instrument monitoring quality assurance to ensure that the point-of-care system is performing according to design parameters. The central site can provide modification of analytical parameters used in the analysis of volatile organic components (VOCs) at the point-of-care system in order to optimize detection of specific diseases. The central site can provide customer management for the point-of-care system, such as instrument monitoring and billing services.

As an application of telemedicine, data from collection of breath, concentration, desorption and analysis steps performed at the remote point-of-care system can be transmitted to the central site via the Internet or telephone. The central site can resolve breath specific features including for example instrument management issues (calibration) and information about the evolving epidemiology of diseases of interest (e.g. new strains of influenza and tuberculosis).

The present invention also provides a standoff breath collection system at the point-of-care system and a method that avoids the use of mouthpieces and the disadvantages associated with breath collection apparatuses requiring mouthpieces. The standoff breath collection system of the present invention provides a donor system having the advantage to collect and analyze a sample of breath without incurring disadvantages, such as the donor need not be conscious; samples can be collected from unconscious or drowsy subjects, the donor need not be cooperative since there is no need to maintain a seal between the lips and the mouthpiece, the donor does not need to wear a nose-clip, there is no resistance to overcome during expiration, and the donor breathes normally while the sample is collected, the risk of contamination of the device with microorganisms is greatly reduced, and collection of a breath volatile organic components (VOCs) sample entails no use of disposable items.

The invention will be more fully described by reference to the following drawings.

### BRIEF DESCRIPTION OF THE DRAWINGS

FIG. 1 is a schematic diagram of a system for remote collection and analysis of volatile organic components (VOCs) in breath.

FIG. 2 is a schematic diagram of a system for remote collection and analysis of volatile organic components (VOCs) in breath including a plurality of point-of-care systems.

FIG. 3 is a schematic view of a donor system used in the breath collection system of the present invention.

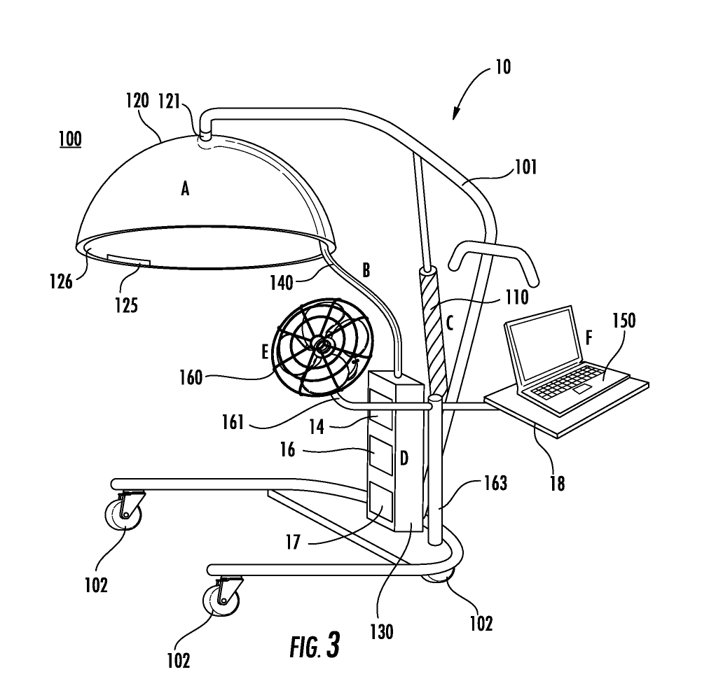

FIG. 4 is a chromatogram of normal human breath analyzed for n-alkanes having different chain lengths using the breath collection system of present invention.

### DETAILED DESCRIPTION OF THE INVENTION

Reference will now be made in greater detail to a preferred embodiment of the invention, an example of which is illustrated in the accompanying drawings. Wherever possible, the same reference numerals will be used throughout the drawings and the description to refer to the same or like parts.

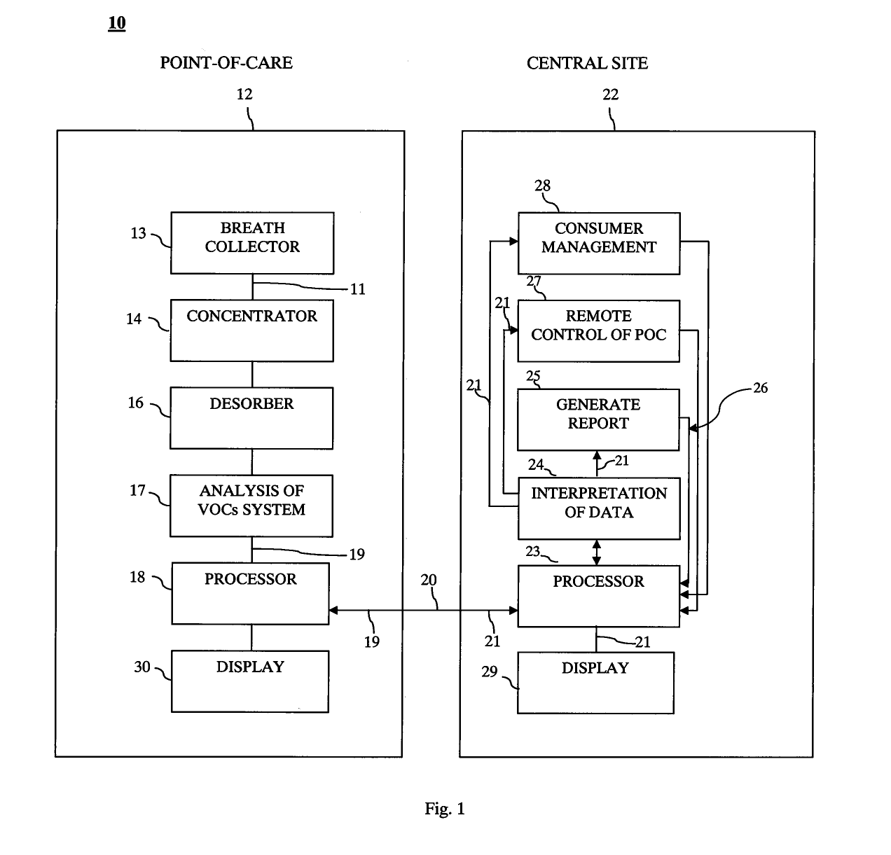

FIG. 1 is a schematic diagram of remote collection and analysis of volatile organic components in breath system 10. Breath collection system 10 includes point-of-care system 12 and central site system 22.

Point-of-care system 12 includes breath collector 13 to collect human breath 11 for analysis. Concentrator 14 receives collected alveolar breath 11. Concentrator 14 can be used to concentrate volatile organic components (VOCs) of breath 11. For example, concentrator 14 can pump a sample of breath through a sorbent trap that contains a resin, for example Tenax or activated carbon, such as Carbotrap or Carbopack. The sorbent trap can be a stainless steel tube containing the resin. Concentrator 14 can include functions for controlling thermodynamic factors such as managing the interaction of water, volatile organic components (VOCs) and condensation. Concentrator 14 can include designs that manage water, volatile organic components (VOCs) and temperature changes post expiration. In one embodiment, concentrator 14 includes heated inlet ports followed by cooled traps and controls to cool surroundings from which warm breath is extracted.

A concentrated volatile organic components (VOCs) sample is thermally desorbed from concentrator 14 in desorption unit 16. Analysis of volatile organic components unit 17 receives volatile organic components (VOCs) which have been desorbed from concentrator 14. Analysis of volatile organic components unit 17 can employ a gas chromatograph (GC) to separate the volatile organic components (VOCs), combined with a detector, such as a surface acoustic wave detector (GC SAW) (Z-nose 4200, commercially available from Electronic Sensor Technology, Newbury Park, Calif.), a flame ionization detector (FID), or a mass spectrometer (GC MS). Volatile organic components (VOCs) can also be analyzed with other detection techniques such as infra-red spectrometry or array detection.

Analysis of volatile organic components unit 17 can also employ several different kinds of array detectors, including for example chemoresistor arrays. The detectors employ the same basic mechanism: different components of the array respond differently to volatile organic components (VOCs) in the sample, so that the combination of signals from the array components generates a unique pattern according to the abundance of different volatile organic components (VOCs) in the sample. Analytical instruments used in analysis of volatile organic components unit 17 can be periodically calibrated e.g. with a calibration standard comprising mixture of analytes with known composition in known quantities, for example n-alkanes.

Processor 18 receives data from analysis of volatile organic components unit 17. Processor 18 can forward data 19 over communications network 20 to central site 22. For example, communications network 20 can include the internet and telephone.

Processor 23 at central site 22 receives data 19. Processor 23 can include interpretation of analytic data module 24 to identify if a pattern of volatile organic components (VOCs) in the sample is consistent with a particular disease. Example diseases include active pulmonary tuberculosis, lung cancer, or breast cancer.

Interpretation of analytic data module 24 can include algorithm learning to refine diagnostic algorithms used in interpretation of analytic data module 24 at central site 22 and improve their accuracy based on accumulated data from collected breath samples 11 at point-of-care system 12 in an ongoing fashion. Data 21 generated by interpretation of analytic data module 24 can be used in generate report module 25, remote control of point-of-care module 27 and consumer management module 28.

Generate report module 25 at central site 22 can generate report 26 including data 21 from interpretation of analytic data module 24. For example, report 26 can include an interpretation of high or low risk of active pulmonary tuberculosis. Report 26 can be transmitted to point-of-care system 12 from central site 22 over communications network 20.

Remote control of point-of-care module 27 can provide instrument monitoring quality assurance to ensure that point-of-care system 12 is performing according to design parameters based on data 21 determined at interpretation of data module 24. Remote control of point-of-care module 27 can provide instrument calibration of instruments or apparatus used in point-of-care system 12 to quantify the abundance of volatile organic components (VOCs) in breath samples 11 analyzed at point-of-care system 12.

Remote control of point-of-care module 27 can provide modification of analytical parameters used in analysis of volatile organic components unit 17 in order to optimize detection of specific diseases. For example, central site 22 can instruct point-of-care system 12 to modify the analytical parameters, such as, for example, to alter the GC temperature ramp for improved detection of a specific disease.

Customer management module 28 can employ data 21 from point-of-care system 12 to generate billing services for services performed at point-of-care system 12 and central site 22. For example, customer management module 28 can generate invoices which are forwarded to point-of-care system 12 from central site 22 over communications network 20. Data 21 from interpretation of analytic data module 24, generate report module 25 and consumer management module 28 can be displayed at display 29 at central site 22 or can be forwarded to point-of-care system 12 to be displayed at display 30.

Embodiments of processor 18 and processor 23 may be implemented in connection with a special purpose or general purpose computer that include both hardware and/or software components.

Embodiments may also include physical computer-readable media and/or intangible computer-readable media for carrying or having computer-executable instructions, data structures, and/or data signals stored thereon. Such physical computer-readable media and/or intangible computer-readable media can be any available media that can be accessed by a general purpose or special purpose computer. By way of example, and not limitation, such physical computer-readable media can include RAM, ROM, EEPROM, CD-ROM or other optical disk storage, magnetic disk storage or other magnetic storage devices, other semiconductor storage media, or any other physical medium which can be used to store desired data in the form of computer-executable instructions, data structures and/or data signals, and which can be accessed by a general purpose or special purpose computer. Within a general purpose or special purpose computer, intangible computer-readable media can include electromagnetic means for conveying a data signal from one part of the computer to another, such as through circuitry residing in the computer.

When information is transferred or provided over a network or another communications connection (either hardwired, wireless, or a combination of hardwired or wireless) to a computer, hardwired devices for sending and receiving computer-executable instructions, data structures, and/or data signals (e.g., wires, cables, optical fibers, electronic circuitry, chemical, and the like) should properly be viewed as physical computer-readable mediums while wireless carriers or wireless mediums for sending and/or receiving computer-executable instructions, data structures, and/or data signals (e.g., radio communications, satellite communications, infrared communications, and the like) should properly be viewed as intangible computer-readable mediums. Combinations of the above should also be included within the scope of computer-readable media.

Computer-executable instructions include, for example, instructions, data, and/or data signals which cause a general purpose computer, special purpose computer, or special purpose processing device to perform a certain function or group of functions. Although not required, aspects of the invention have been described herein in the general context of computer-executable instructions, such as program modules, being executed by computers, in network environments and/or non-network environments. Generally, program modules include routines, programs, objects, components, and content structures that perform particular tasks or implement particular abstract content types. Computer-executable instructions, associated content structures, and program modules represent examples of program code for executing aspects of the methods disclosed herein.

Embodiments may also include computer program products for use in the systems of the present invention, the computer program product having a physical computer-readable medium having computer readable program code stored thereon, the computer readable program code comprising computer executable instructions that, when executed by a processor, cause the system to perform the methods of the present invention.

Breath collector 13 and concentrator 14 can be a valved collection unit including a mouth piece. A suitable breath collector is described in U.S. Pat. No. 6,726,637. In one embodiment, features of the breath collection apparatus described in U.S. Pat. Nos. 5,465,728 and 6,725,637 hereby both incorporated by reference into this application can be used for collection, concentration, desorption and analytics of volatile organic components (VOCs).

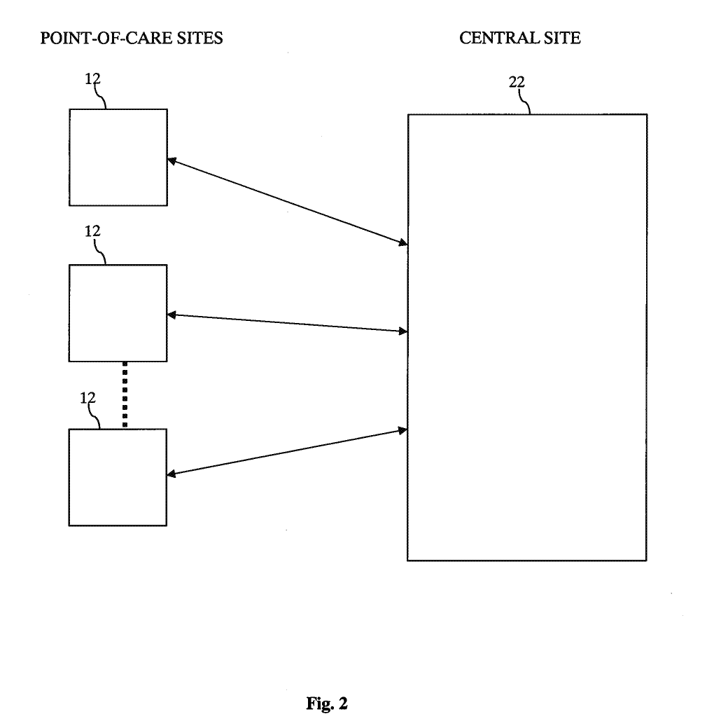

A plurality of point-of-care systems 12 can interface to central site 12, as shown in FIG. 2. Each of point-of-care systems 12 can provide telemedicine for patients at the respective point-of-care systems 12.

Alternatively, point-of-care system 12 includes dome system 100 including the following main components: collection dome 120, tube linking apex of dome interior to analytical system 140, hydraulic lifter (hand operated) 110, analytical system 130, room air fan 160 and laptop computer element 150.

Collection dome 120 can comprise a clear acrylic hemisphere. A suitable collection dome is about 24 inches to about 48 inches in diameter, for example about 38 inches (91 cm) in diameter.

Suspension frame 101 supports collection dome 120 and ancillary apparatus comprising a hydraulic patient lifter 110 (commercially available from Invacare Corporation, Elyria, Ohio). Suspension frame 101 can include wheels 102.

Fan 160 supported by fan support 161 is attached to suspension frame 101 with support 163. Fan 160 can comprise a 10 inch (24 cm) diameter fixed room air fan (commercially available from Vornado Air Circulation Systems Inc., Andover, Kans.).

Analytical system 130 can include concentrator 14, desorber 16, and analysis of volatile organic components unit 17 surface acoustic wave detector (GC SAW). Laptop computer element 150 can include processor 18. Laptop computer 150 can be used for controlling the fan 160. Laptop computer 150 can be attached to computer support 164. Fan support 161 and computer support 164 can be attached or integral with support 163.

Tubing 140 can link a sampling point at apex 122 of dome 120 to the intake of concentrator 14 on analysis of volatile organic components unit 17, such as GC/SAW housed in analytical system 130. Tubing 140 can be comprised of a non-reactive and chemically clean tubing Tygon®.

Computer 150 is employed to control sample collection by activation of fan 160, and to control collection, concentration and analysis of the breath volatile organic components (VOCs) sample in analytical system 130. Computer 150 can also be employed as a remote collection and analysis of volatile organic components (VOCs) in breath to upload chromatographic data and instrument data to a central site 22 via the internet, and to download interpretations of the data to the user and the patient.

An ultraviolet disinfection lamp 125 can be incorporated into dome 120 to sterilize interior surface 126 of dome 120 after use. This precaution need only be employed in a high-burden country where pulmonary tuberculosis and other infectious diseases are endemic.

Remote collection and analysis of volatile organic components (VOCs) in breath system 10 including dome system 100 can be operated as follows:

1. Positioning of the dome: Dome system 100 is wheeled to the point-of-use so that collection dome 120 is positioned centrally over the breath donor's head. The breath donor may be seated comfortably, though collection dome 120 may also be employed when the breath donor is standing, or lying recumbent. When the breath donor is standing or sitting, collection dome 120 is lowered to a point where its lower edge is parallel with the shoulders. When the breath donor is lying recumbent, the collection dome 120 is lowered to a point where its lower edge is approximately one inch (2 to 3 cm) above the chest.

2. Positioning of the fan: Fan 160 is positioned to blow near vertically into collection dome 120 in order to replace its contents with room air.

3. Air sample collection and analysis:

(a) Time zero to 2.0 minutes: Fan 160 and analytical system 130 including concentrator 12 are both switched on. During this period, collection dome 120 is constantly flushed with room air so that all breath volatile organic components (VOCs) are expelled. The concentrator 12 collects room air from apex 122 of collection dome 120 at 35 ml/min for about 2.0 min. Fan 160 and analytical system 130 including pre-concentrator pump are both switched off at the end of the 2.0 minute period.

(b) Time: 2.0 to 7.0 minutes: Desorber 16 is switched on, and a stream of pure helium is introduced into the sorbent trap of desorber 16 in order to rapidly flush the concentrated volatile organic components (VOCs) from the room air sample on to a column of the GC at analysis of volatile organic components unit 17. The volatile organic components (VOCs) are analyzed by fast GC/SAW, as the column temperature is ramped from ambient temperature to 220° C. over a period of about 2.0 min in analysis of volatile organic components unit 17. The GC column is then cooled to ambient temperature.

During this period, breath volatile organic components (VOCs) accumulate at apex 122 of collection dome 120. The breath donor should not engage in activities that might modify the composition of breath volatile organic components (VOCs), such as tobacco smoking, eating or drinking. However, other activities such as speaking, reading or watching television will not affect the accumulation of the breath sample at apex 122 of collection dome 120.

4. Breath sample collection and analysis:

Time: 7.0 to 9.0 minutes. Concentrator 12 in analysis of volatile organic components unit 17, such as GC/SAW, is switched on again, and the process of collection, concentration and analysis is repeated as described above. However, the sample now comprises breath from apex 122 of collection dome 120 instead of room air. At about 9.0 minutes, collection dome 120 is raised and the breath donor may leave,

5. Data transmission: Chromatographic data analysis of volatile organic components unit 17 can be transmitted from the point-of-care system 12 to a central site 22 over communications network 20 via the internet or a telephone link, as shown in FIG. 1.

6. Calibration of the chromatogram: Concentrator 12 in analysis of volatile organic components unit 17, such as GC/SAW.GC/SAW is calibrated at the beginning of each day of usage by injecting a known quantity of a standard solution containing a mixture of volatile organic components (VOCs) in known concentrations, for example, a mixture of standard n-alkanes. The abundance of a volatile organic components (VOCs) peak observed in a chromatogram of breath or air is normalized to the standard i.e. V_{b}/I_{b} where V_{b} denotes the area under the curve (AUC) detected by GC/SAW in breath, and I_{b} denotes the AUC of the chromatographic peak associated with the internal standard.

7. Analysis of data: The alveolar gradient of each volatile organic components (VOCs) peak (i.e. abundance in breath minus abundance in ambient room air) can be determined as alveolar gradient=V_{b}/I_{b}−Vₐ/Iₐ Vₐ and where Vₐ and Iₐ denote corresponding values derived from the associated sample of room air. The polarity of the alveolar gradient varies with the kinetics of volatile organic components (VOCs) metabolism: its value is positive when synthesis is greater than clearance, and negative when clearance is greater than synthesis.

### Physiological Basis of the Invention

Human breath is excreted at body temperature, about 36.7° C. In an air-conditioned environment, ambient room air is cooler (usually about 20-25° C.). Hence, expired breath rises upwards in an air-conditioned environment because it is displaced by the cooler denser room air. The physiological basis of dome system 100 is to capture expired breath into a container as it is displaced upwards. The container employed in this invention is collection dome 120 including an inverted hemispherical bowl constructed of clear acrylic plastic. However, containers of different sizes and shapes may fulfill the same function equally well. The captured breath is then pumped from apex 122 of dome 120 to collector 12 including a volatile organic components (VOCs) trap.

### Analysis of the Collected Sample

The volatile organic components (VOCs) trap may contain a sorbent resin (e.g. Tenax OD) or activated carbon (e.g. Carbotrap 0). The volatile organic components (VOCs) trap may be removed, in order to analyze the sample at another site, or else the sample may be thermally desorbed by desorber 14 for analysis at the point-of-care system 12.

Results in humans: The device was evaluated in normal human subjects. The analytical system comprised a pre-concentrator (a sorbent trap containing Tenax 0), a thermal desorber, and a gas chromatograph with a surface acoustic wave detector (GC/SAW). A laptop computer controlled the collection and analysis procedure. The human trial was conducted following the detailed procedure described above under “Usage of the device”.

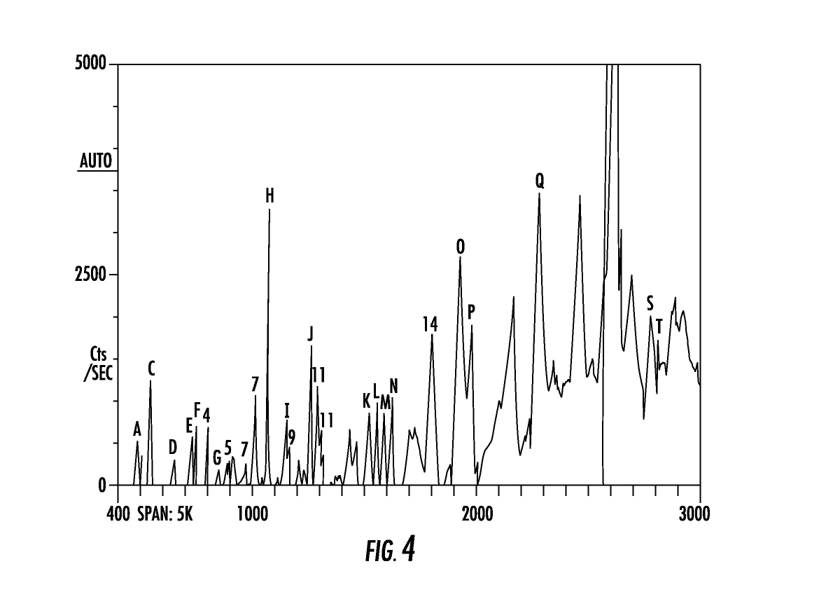

Pursuant to the human trial, a representative chromatogram of human breath volatile organic components (VOCs) was obtained and is shown in FIG. 4. As shown in FIG. 4, each peak represents a different volatile organic component (VOC) in the breath sample of the subject, and the area under curve of the peak varies with the abundance of the volatile organic components (VOCs) in the breath sample. The x-axis is the scan number. Typically, the chromatogram is generated in approximately one minute. Peaks are labeled by automated recognition software, In this case, peaks identified by letters of the alphabet represent volatile organic components (VOCs) whose retention times are similar to those previously programmed for identification (n-alkanes with different chain lengths), while the numbered peaks are all other volatile organic components (VOCs). A volatile organic components (VOCs) peak may be identified qualitatively by its retention time, and then quantified by its area under curve.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A telemedicine system for collection and analysis of volatile organic components in alveolar breath comprising:
a point-of-care system, said point-of-care system includes a breath collector to collect a sample of the alveolar breath, means for analysis of said collected breath to generate breath data of the volatile organic components contained in the alveolar breath and means for communicating with a central site to forward the breath data to said central site, and
said central site including means for receiving said breath data and means for interpreting said breath data to identify if a pattern of the volatile organic components is consistent with a particular disease,
wherein said central site forwards the interpreted data to said point-of-care system.

2. The telemedicine system of claim 1 wherein said point-of-care system further comprises a concentrator for concentrating the volatile organic components of the alveolar breath and a desorber for desorbing the volatile organic components, wherein the desorbed volatile organic components are analyzed in said means for analysis of said collected breath.

3. The telemedicine system of claim 1 wherein the means for analysis of said collected breath includes a surface acoustic wave detector (GC SAW).

4. The telemedicine system of claim 1 wherein the means for interpreting said breath data includes algorithm learning to refine diagnostic algorithms used in means for interpreting said breath data.

5. The telemedicine system of claim 1 wherein said central site includes a generate report module to generate report of the interpretation of data from the means for interpreting said breath data, said report being transmitted from said central site to said point-of-care system.

6. The telemedicine system of claim 1 wherein said central site includes means for remote control of the point-of-care system to provide instrument monitoring quality assurance to ensure the point-of-care system is performing according to predetermined parameters.

7. The telemedicine system of claim 6 wherein the means for remote control of the point-of-care system calibrates instruments or apparatus used in the point-of-care system to quantify the abundance of the volatile organic components (VOCs) in the breath sample.

8. The telemedicine system of claim 6 wherein the means for remote control of the point-of-care system determines analytical parameters used in the means for analysis of said collected breath analysis to optimize detection of specific diseases.

9. The telemedicine system of claim 1 wherein said central site includes means for
customer management to determine customer management from the breath data and the interpreted data.

10. The telemedicine system of claim 1 wherein the means for
customer management provides billing services.

11. The telemedicine system of claim 1 wherein the breath collector comprises a dome system, said dome system including a collection dome.

12. The telemedicine system of claim 11 wherein said collection dome is a clear acrylic hemisphere.

13. The telemedicine system of claim 11 wherein said collection dome is coupled to a concentrator for concentrating the volatile organic components of the alveolar breath with tubing.

14. The telemedicine system of claim 11 wherein said dome system further comprises
a fan positioned below said collection dome for flushing said collection dome.

15. A method for remote collection and analysis of volatile organic components in alveolar breath comprising the steps of:
collecting a sample of the alveolar breath at a point-of-care;
generating breath data of the volatile organic components contained in the alveolar breath;
communicating with a central site to forward the breath data to said central site; and
interpreting said breath data at said central site for identifying if a pattern of the volatile organic components is consistent with a particular disease at the central site to determine interpreted data; and
forwarding the interpreted data to said point-of-care.

16. The method of claim 15 wherein said step of
interpreting said breath data includes algorithm learning to refine diagnostic algorithms used for interpreting said breath data.

17. The method of claim 15 further comprising the steps of generating a report of the interpretation of said breath data and transmitting said report from said central site to said point-of-care.

18. The method of claim 15 further comprising the step of controlling of the point-of-care at said central site to provide instrument monitoring quality assurance to ensure the point-of-care is performing according to predetermined parameters.

19. The method of claim 15 further comprising the step of controlling of the point-of-care at said central site to calibrate instruments or apparatus used in the point-of-care to quantify the abundance of the volatile organic components (VOCs) in the breath sample.

20. The method of claim 15 further comprising the step of controlling of the point-of-care at said central site to determine analytical parameters used in the collecting step to optimize detection of specific diseases.

21. The method of claim 15 further comprising the step of providing billing services to the point-of-care from the central site, the billing services being based on the breath data and the interpreted data.

22. The method of claim 15 wherein the collecting step comprises collecting the breath with a dome system, said dome system including a collection dome.
